(19) United States
(12) Patent Application Publication
(10) Pub. No.: US 2023/0382830 A1
(43) Pub. Date: Nov. 30, 2023

(54) Selective Lightly Branched Alcohols Through Hydroformylation Of Isomerized Linear Olefin Feeds

(71) Applicants: Zsigmond Varga, Schaerbeek (BE); Sina Sartipi, Willebroek (BE); Silvio Carrettin, Kraainem (BE); Nele Viggria, Hombeek (BE); Hans Goris, Zaventem (BE)

(72) Inventors: Zsigmond Varga, Schaerbeek (BE); Sina Sartipi, Willebroek (BE); Silvio Carrettin, Kraainem (BE); Nele Viggria, Hombeek (BE); Hans Goris, Zaventem (BE)

(21) Appl. No.: 18/245,920

(22) PCT Filed: Sep. 29, 2021
(86) PCT No.: PCT/EP2021/076721

§ 371 Date: Mar. 20, 2023

Related U.S. Application Data
(60) Provisional application No. 63/089,479, filed on Oct. 8, 2020.

(51) Int. Cl. C07C 29/141; C07C 45/50; C07C 5/22

(52) U.S. Cl. CPC C07C 29/141; C07C 45/505; C07C 5/222

(57) ABSTRACT

This disclosure relates to a primary alcohol composition of linear and branched C₁₁, C₁₃, C₁₅, C₁₇, C₁₉ or C₂₁ primary alcohols, wherein at least 50% of the branched alcohol chains are mono-branched chains with a branch at the second carbon atom relative to the hydroxyl carbon. Branching is selective and has been found to be preferably at least 80% in the 2-carbon position (the beta carbon) of the alcohol. This disclosure also relates to a process using an isomerized linear alpha olefin (LAO) as a feed for hydroformylation to produce the lightly branched C₁₁, C₁₃, C₁₅, C₁₇, C₁₉ or C₂₁ primary alcohols.

### FIELD

This application relates to an alcohol composition and a process for preparing the same, wherein the alcohol composition comprises one or more linear and branched C₁₁, C₁₃, C₁₅, C₁₇, C₁₉ or C₂₁ primary alcohols and wherein at least 50% of the branched alcohol chains are mono-branched, with a branch at the second carbon atom relative to the hydroxyl carbon. In particular this application relates to lightly branched tridecanols or lightly branched pentadecanols that contain predominantly mono-branched chains with a branch at the second carbon atom relative to the hydroxyl.

### BACKGROUND

Both linear and branched primary alcohols may be converted to one or more derivatives that can be used in laundry detergents, cleaning products, as personal care ingredients, as emulsifying agents or as a lubricant additive. The alcohols can be obtained through oxo hydroformylation of an olefin. Oxo hydroformylation may take place by contacting syngas, a mixture comprising carbon monoxide and hydrogen, with an olefin in the presence of a metal catalyst to form a hydroformylation reaction product.

Exxal™ (ExxonMobil) alcohols are isomeric branched, primary alcohols that contain both even- and odd-numbered hydrocarbon chains, ranging from C₈ to C₁₃. Exxal™ alcohols have been used to synthesize derivatives used in surfactants, polymer additives, adhesives, fuel additives and lubricants. They have also been used as solvents or co-solvents for coatings, inks and metal extraction in mining.

NEODOL™ (Shell) alcohols are highly linear primary alcohols that typically contain 75-85% by weight linear alcohols.

U.S. Pat. No. 5,849,960 discloses a highly branched primary alcohol composition having 8 to 36 carbon atoms and an average number of branches per molecule chain of at least 0.7. In a preferred embodiment, the average number of branches per molecule chain ranges from 1.5 to 2.3. As determined by NMR, Table 1 of U.S. Pat. No. 5,849,960 discloses that NEODOL™ 45, a C₁₄₋₁₅ alcohol, has only 18.8 wt % branching at the C₂ atom of the alcohol. Further, as determined by gas chromatography (GC), NEODOL™ 45 is disclosed to be 78% linear alcohol.

U.S. Pat. No. 7,183,446 discloses an alcohol mixture substantively comprising alcohols having 13 or 15 carbon atoms, where the mixture comprises 40 to 60% by weight of linear alcohols, from 30 to 40% by weight of 2-methyl-branched alcohols and from 2 to 7% by weight of 2-ethyl-branched alcohols. U.S. Pat. No. 7,183,446 defines an alcohol mixture as a mixture which has at least 2, preferably at least 3, different alcohols.

U.S. Pat. No. 8,962,541 discloses C₄ to C₁₅ alcohol compositions comprising conjugated unsaturated carbonyl compounds. In addition, the C₄ to C₁₅ alcohol, the compositions may comprise alcohols having different carbon numbers.

U.S. Pat. No. 9,828,565 discloses a mixture of tridecanols where at least about 60 wt % of the mixture is linear tridecanol.

U.S. Pat. No. 9,828,573 discloses a composition comprising a mixture of pentadecanols wherein at least about 60 wt % of the mixture is linear pentadecanol and at least about 10 wt % of the mixture is branched pentadecanols wherein the branched pentadecanols have branching on the second carbon atom.

There is a renewed interest in using lightly branched C₁₁, C₁₃, C₁₅, C₁₇, C₁₉ or C₂₁ alcohols and their derivatives as surface active agents, esters and additives. Accordingly, there is a need for a process to produce an alcohol with an increased branched content, showing selective branching, that contains predominantly mono-branched chains with a branch at the second carbon atom relative to the hydroxyl carbon.

### SUMMARY

Disclosed is a primary alcohol composition comprising linear and branched Cₙ alcohol chains, wherein at least 50% of the branched alcohol chains are mono-branched with a branch at the second carbon atom relative to the hydroxyl carbon, where n is an odd integer, taking one or more values ranging from 11 to 21. Preferably, the primary alcohol composition is lightly branched, having an average number of branches per molecule chain less than 1.4. In preferred embodiments, at least 80% of the branched alcohol chains are mono-branched with a branch at the second carbon atom. In preferred embodiments, the average number of branches per molecule chain is greater than 0.4, optionally greater than 0.6, yet optionally greater than 0.7. In preferred embodiments, the primary alcohol composition comprises less than 60 wt %, preferably less than 45 wt % linear alcohol, and more preferably less than 5 wt % linear alcohol.

Further disclosed is a process for preparing a primary alcohol composition comprising linear and branched Cₙ alcohols, the process comprising: (a) isomerization of a C₍ₙ₋₁₎ linear alpha olefin feed to produce an C₍ₙ₋₁₎ isomerized olefin, wherein n is an odd integer, taking one or more values ranging from 11 to 21, (b) contacting the isomerized olefin with syngas and a hydroformylation catalyst, (c) hydrogenating the reaction mixture of step (b) and (d) harvesting the primary alcohol composition comprising linear and branched of Cₙ alcohol chains, wherein at least 50% of the branched alcohol chains are mono-branched, with a branch at the second carbon atom relative to the hydroxyl carbon of the Cₙ alcohol chain. Preferably, the primary alcohol composition is lightly branched, having an average number of branches per molecule chain less than 1.4. In preferred embodiments, at least 80% of the branched alcohol chains are mono-branched with a branch at the second carbon atom. In preferred embodiments, the average number of branches per molecule chain is greater than 0.4, optionally greater than 0.6, yet optionally greater than 0.7. In preferred embodiments, the primary alcohol composition comprises less than 60 wt %, preferably less than 45 wt %, and more preferably less than 5 wt % linear alcohol.

Also disclosed is a composition comprising one or more derivatives of the primary alcohol composition, wherein the derivative comprises esters of dicarboxylic acids, esters of polycarboxylic acids, alkoxylated alcohols, sulfated alcohols, sulfated alkoxylated alcohols and alcohol ether amines. Alternatively, the derivative comprises esters of the primary alcohol composition with one or more acids such as phthalic acid, adipic acid, sebacic acid, lauric acid, myristic acid, palmitic acid, stearic acid, oleic acid, succinic acid and trimellitic acid. Alternatively, the derivative comprises phosphites of low volatility to be used as polymer stabilizers.

### BRIEF DESCRIPTION OF THE FIGURES

For a better understanding of the nature, objects, and processes involved in this disclosure, reference should be made to the detailed description taken in conjunction with the accompanying FIG. 1. The subject matter disclosed is capable of considerable modifications, alterations, combinations, and equivalents in form and function, as will occur to those skilled in the art and having the benefit of this disclosure.

FIG. 1 discloses the on stream composition of isomerized olefin Feed I and Feed II showing on the y axis the conversion of linear alpha olefin (LAO), the yields to linear internal olefin (LIO), branched olefin (BO) and dimer as a function of catalyst hours on stream on the x axis (Time On Stream). The circles in FIG. 1 are directed to the conversion of LAO, the triangles are directed to the yield of the linear internal olefin, the squares are directed to the yield of the branched olefin and the diamonds are directed to the yield of dimers.

### DETAILED DESCRIPTION

As used herein, “wt %” means percentage by weight, “vol %” means percentage by volume, “mol %” means percentage by mole, “ppm” means parts per million. All “ppm” as used herein are ppm by weight unless specified otherwise. All concentrations herein are expressed on the basis of the total amount of the composition in question. All ranges expressed herein should include both end points as two specific embodiments unless specified or indicated to the contrary.

As used herein, the term “alcohols having different carbon numbers” means alcohols with carbon chains of different length. For example, a mixture comprising a C₁₄ and a C₁₅ alcohol would be a mixture of alcohols having different carbon numbers.

As used herein, the term “a branch at the second carbon” means a branch at the second carbon or the 3 carbon of the primary alcohol. The first carbon or the primary carbon is the C₁ carbon with the hydroxyl group. The primary carbon may also be known as the hydroxyl carbon. The second carbon or the C₂ carbon is adjacent to the primary carbon of the primary alcohol.

As used herein, the term “Cₙ₋₁ linear alpha olefin” means a linear alpha olefin where n is an odd number (integer) ranging from 11 to 21. As defined herein, the term “Cₙ alcohol” means a primary alcohol where n is an odd number (integer) ranging from 11 to 21. As used herein, the term “primary alcohol” is an alcohol which has the hydroxyl group connected to a primary carbon atom or the C₁ carbon.

As used herein, the term “lightly branched alcohol” means an alcohol containing linear and branched chains having an average number of branches per molecule chain which is less than 1.4.

As used herein, the term linear alpha olefin (LAO) is a linear olefin where the double bond of the olefin is between the first and second carbon of the olefin.

As used herein, the term linear internal olefin (LIO) is a linear olefin where the double bond is located anywhere between the second and second-to-last carbon of the olefin. In other words, the term linear internal olefin describes any linear olefin with a double bond which is not located at the primary (or alpha) position.

As used herein, the term “olefin” may refer to a branched or unbranched unsaturated hydrocarbon having one or more carbon-carbon double bonds. The term “olefin” is intended to embrace all structural isomeric forms of an olefin.

As used herein, the term “selective branching” means that there is a substantial fraction of branching in a specific location along the chain, i.e. at the second carbon position of the alcohol. The term “selective branching” also encompasses branching at other positions of the carbon backbone of the alcohol. However, in the method of the invention, the total branching at these other positions of the carbon backbone is not more than 50%, preferably not more than 20%, more preferably not more than 10%.

As used herein, the term “predominantly branched at the second carbon atom of the alcohol” means that about 80% to about 100% of the branched alcohols comprised in the alcohol composition are branched at the second carbon atom.

Branched primary alcohols, including pentadecanols, may be converted to one or more derivatives that can be used in laundry detergents, cleaning products, as personal care ingredients, as emulsifying agents or as a lubricant additive. Linear 1-pentadecanol can be obtained through oxo hydroformylation of linear tetradecene. The latter procedure yields a pentadecanol mixture with up to about 34 wt % branched alcohol content.

There is a renewed interest in using branched alcohols and their derivatives as surface active agents, esters and additives. Without wishing to be bound by theory, it is believed that the average degree of branching in the hydrophilic backbone of the alcohol imparts additional functionality when compared to the linear alcohol. In one instance, the branched alcohols have shown increased cold water solubility and/or surfactant performance. Accordingly, there is a need for a process to produce an alcohol composition which is lightly branched, having an average degree of branching below 1.4 and therefore exhibiting good biodegradability and, at the same time containing enough branched species, preferably more than 40 wt % of the total alcohol content, which ensures increased cold water solubility and/or surfactant performance. Furthermore there is a need for methods that can tailor the geometry of the alcohol molecular chain to thereby adjust the properties of the derivative obtained therefrom, for example adjust the hydrophobic character and/or improving of the detergency of the surfactants obtained therefrom.

Olefin Feed

In the formation of a composition containing linear and branched C₁₁, C₁₃, C₁₅, C₁₇, C₁₉ or C₂₁ primary alcohols, isomerization of the linear alpha olefin (LAO) feed is first performed, resulting in an isomerized olefin feed. In one embodiment, the isomerization reaction yields an isomerized olefin feed rich in linear internal olefin (LIO). As used herein, an olefin feed rich in linear internal olefin means an olefin feed comprising 50 wt % or more linear internal olefins from to the total olefin content. In another embodiment, isomerization reaction yields an isomerized olefin feed rich in branched olefin (BO). As used herein, an olefin feed rich in branched olefin means an olefin feed comprising 50 wt % or more branched olefins from the total olefin content. The isomerization of the linear olefin feed to linear internal olefins and branched olefins is performed by methods well known to the person skilled in the art or by streaming the olefin feed over a zeolite catalyst.

The olefin feed and the isomerized olefin feed according to the present invention can be any C₍ₙ₋₁₎H₂₍ₙ₋₁₎, where C is a carbon atom, H is a hydrogen atom and n is an odd number of carbon atoms ranging from n=11 to 21. The term “olefin” may refer to a branched or unbranched unsaturated hydrocarbon having one or more carbon-carbon double bonds. The term “olefin” is intended to embrace all structural isomeric forms of an olefin. Examples of olefins include decene (C₁₀H₂₀), dodecene (C₁₂H₂₄), tetradecene (C₁₄H₂₈), hexadecane (C₁₆H₃₂) and octadecene (C₁₈H₃₆).

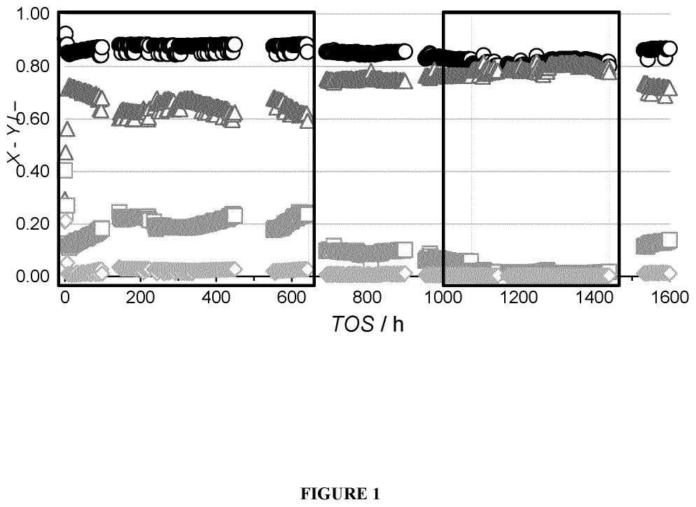

Two gas chromatographic (GC) methods were employed to characterize the olefin feeds: one for measuring the linear alpha olefin (LAO) content and the second for measuring the branched olefin (BO) content of the feed. The linear and branched content, which were inferred from the GC data are disclosed in FIG. 1. Specifically, FIG. 1 presents the on stream composition of two isomerized C₁₄ olefin feeds showing the conversion of linear alpha olefin to linear internal olefin, branched olefin and dimer. The circles of FIG. 1 are directed to the conversion of LAO, the triangles are directed to the yield of the linear internal olefin (LIO), the squares are directed to the yield of the branched olefin (BO) and the diamonds are directed to the yield of the dimer.

In one embodiment, the olefin feed is substantially a linear C₁₀ olefin. The isomerized C₁₀ olefin feed is a mixture of linear internal and branched C₁₀ olefins.

In another embodiment, the olefin feed is substantially a linear C₁₂ olefin. The isomerized C₁₂ olefin feed is a mixture of linear internal and branched C₁₂ olefins.

In one embodiment, the olefin feed is substantially a C₁₄ olefin. The isomerized C₁₄ olefin feed is a mixture of linear internal and branched C₁₄ olefins. In a further embodiment, the isomerized C₁₄ olefin feed comprises from about 40 to about 90 wt % linear internal olefins and from 0 to about 50 wt % branched olefins.

In another embodiment, the olefin feed is substantially a C₁₆ olefin. The isomerized C₁₆ olefin feed is a mixture of linear internal and branched C₁₆ olefins.

In another embodiment, the olefin feed is substantially a C₁₈ olefin. The isomerized C₁₈ olefin feed is a mixture of linear internal and branched C₁₈ olefins.

In another embodiment, the olefin feed is substantially a C₂₀ olefin. The isomerized C₂₀ olefin feed is a mixture of linear internal and branched C₂₀ olefins.

Isomerization of the olefin feed is performed by methods well known to the person skilled in the art or by streaming the olefin feed over a zeolite catalyst. In embodiments of the invention, isomerization of the olefin feed resulting in a mixture of linear internal and branched olefins occurs under the following reaction conditions: temperature about 100° C. to about 180° C. and pressure about 1 to about 2 barg. The olefin feed is supplied at a weight hourly space velocity (WHSV) from about 1 to about 10 h⁻¹.

In preferred embodiments of the invention, the pressure during the isomerization is about 1.5 barg. The temperature during the isomerization ranges from about 140° C. to about 160° C., alternatively from about 140° C. to about 150° C.

In embodiments of the invention, the olefin feed is supplied at a weight hourly space velocity (WHSV) from about 5 to about 10 h⁻¹. In a preferred embodiment, the weight hourly space velocity is about 5 h⁻¹.

The isomerization catalyst can be chosen from a family of zeolites, typically containing 10-membered rings, including but not limited to ZSM-48. In embodiments of the invention, the isomerization catalyst is a molecular sieve or zeolite. Molecular sieves or zeolites are hydrated aluminosilicate minerals made from interlinked tetrahedra of alumina (AlO₄) and silica (SiO₄). Suitable catalysts comprise microporous crystalline aluminosilicates selected from the group consisting of ZSM-5, ZSM-23, ZSM-35, ZSM-11, ZSM-12, ZSM-48, ZSM-57, and mixtures or combinations thereof. In one embodiment, the isomerization catalyst is a microporous crystalline aluminosilicate. In another embodiment, the isomerization catalyst is selected from the group consisting of ZSM-5, ZSM-23, ZSM-35, ZSM-11, ZSM-12, ZSM-48, ZSM-57, and mixtures or combinations thereof. In a further embodiment, the zeolite catalyst is ZSM-48.

In alternative embodiments, the isomerization catalyst comprises a mesoporous material having a collidine uptake of greater than about 100 μmol g⁻¹.

In embodiments of the invention, the microporous crystalline aluminosilicate isomerization catalyst has a SiO₂/Al₂O₃ molar ratio of less than or equal to about 100.

In a further embodiments of the invention, the SiO₂/Al₂O₃ ratio of the zeolite catalyst ranges from about 100 to about 75, preferably from about 60 to about 90, more preferably from about 65 to about 75.

In one embodiment the SiO₂/Al₂O₃ ratio of the zeolite isomerization catalyst is about 70.

In another embodiment, the SiO₂/Al₂O₃ ratio of the zeolite catalyst ranges from about 85 to about 95. In a further embodiment, the SiO₂/Al₂O₃ ratio of the zeolite isomerization catalyst is about 90.

Primary Alcohol Composition

In one embodiment, a primary alcohol composition according to the present invention comprises linear and branched C₁₁, C₁₃, C₁₅, C₁₇, C₁₉ or C₂₁ alcohols, wherein at least 50% of the branched alcohol chains are mono-branched with a branch at the second carbon atom relative to the hydroxyl carbon. In a further embodiment, the alcohol composition comprises linear and branched alcohol chains, the branched alcohol chains are predominantly mono-branched with a branch at the second carbon atom of the alcohol. In a further embodiment, about 80% to about 100% of the branched alcohol chains are mono-branched chains with a branch at the second carbon atom. In an even further embodiment, about 90% to about 100% of the branched alcohol chains are mono-branched chains with a branch at the second carbon atom.

In a further embodiment, the composition comprises one or more of C₁₁, C₁₃, C₁₅, C₁₇, C₁₉ or C₂₁ alcohols wherein at least 80% of the branched alcohol chains are mono-branched chains branched at the second carbon atom. In a further embodiment, the composition of the invention is characterized by at least 90% of the branched alcohol chains that are mono-branched chains branched at the second carbon atom. In a further embodiment, the composition of the invention is characterized by about 93% of the branched alcohol chains that are mono-branched chains branched at the second carbon atom. In a further embodiment, the composition of the invention is characterized by about 99% of the branched alcohol chains that are mono-branched chains branched at the second carbon atom.

In one embodiment, a primary alcohol composition comprises linear and branched Cₙ alcohols, wherein at least 50% of the branched alcohol chains are mono-branched chains with a branch at the second carbon atom relative to the hydroxyl carbon, where n is an odd integer, taking one or more values ranging from 11 to 21. In a further embodiment, the primary alcohol composition comprises linear and branched Cₙ alcohol, with at least 80% of the branched chains being mono-branched chains at the second carbon atom. In a further embodiment, the primary alcohol composition comprises linear and branched Cₙ alcohol, with at least 90% of the branched chains being mono-branched chains at the second carbon atom.

In a further embodiment, the primary alcohol composition of the present invention does not comprise alcohols having different carbon numbers.

To characterize the alcohol compositions of the present invention, gas chromatographic (GC) and nuclear magnetic resonance (NMR) methods were employed. Two NMR methods were employed to characterize the branching in the alcohol samples. One method utilized ¹H NMR to determine the average number of branches per molecule chain (also referred herein as the degree of branching or branching index) and the second method utilized ¹³C NMR to determine the branch site distribution, i.e. the percent of branching in the 2-, 3-, 4- and 5+-carbon positions of the alcohol.

In one embodiment, the alcohol composition comprises lightly branched Cₙ primary alcohols, wherein n is an odd number ranging from 11 to 21.

In one embodiment, the Cₙ alcohol displays less than an average number of 1.3 branches per molecule chain. In a further embodiment, the Cₙ alcohol displays less than an average number of 1.2 branches per molecule chain. In a further embodiment, the Cₙ alcohol displays less than an average number of 1.0 branch per molecule chain. In a further embodiment, the Cₙ alcohol displays less than an average number of 0.8 branch per molecule chain. In a further embodiment, the Cₙ alcohol displays an average number of about 0.5 to about 0.8 branches per molecule chain.

In embodiments of the invention, the primary alcohol composition comprises linear and branched Cₙ alcohols, wherein n is equal to one or more of 11, 13, 15, 17, 19 or 21, wherein the composition is lightly branched, with an average number of branches per molecule chain of less than 1.4. In a further embodiment, the primary alcohol composition of the present invention is lightly branched with an average number of branches per molecule chain greater than 0.4, optionally greater than 0.6, optionally greater than 0.7.

In embodiments of the invention, the primary alcohol composition of the present invention comprises less than 60 wt %, preferably less than 45 wt % linear Cₙ alcohol, more preferably less than 5 wt % linear Cₙ alcohol.

In one embodiment, the alcohol composition is lightly branched and comprises linear and branched C₁₁ primary alcohols substantially branched at the second carbon atom.

In another embodiment, the alcohol composition is lightly branched and comprises linear and branched C₁₃ primary alcohols substantially branched at the second carbon atom.

In another embodiment, the alcohol composition is lightly branched and comprises linear and branched C₁₅ primary alcohols substantially branched at the second carbon atom.

In another embodiment, the alcohol composition is lightly branched and comprises linear and branched C₁₇ primary alcohols substantially branched at the second carbon atom.

In another embodiment, the alcohol composition is lightly branched and comprises linear and branched C₁₉ primary alcohols substantially branched at the second carbon atom.

In another embodiment, the alcohol composition is lightly branched and comprises linear and branched C₂₁ primary alcohols substantially branched at the second carbon atom.

In another embodiment, the primary alcohol composition of the present invention comprises linear and branched Cₙ alcohols, wherein the linear and branched Cₙ alcohol is converted from isomerized a C₍ₙ₋₁₎ linear alpha olefin.

In another aspect of the invention, a composition is disclosed comprising one or more derivatives of the primary alcohols of the present invention. In a further embodiment, the derivative of the primary alcohol comprises esters of dicarboxylic acids, esters of polycarboxylic acids, alkoxylated alcohols, sulfated alcohols, sulfated alkoxylated alcohols and alcohol ether amines

In another embodiment, the derivative comprises esters of the primary alcohol composition with one or more acids. In a further embodiment, the acids comprise phthalic acid, adipic acid, sebacic acid, succinic acid and trimellitic acid.

In another embodiment, the derivative comprises phosphites of low volatility to be used as polymer stabilizers.

In one embodiment, the primary alcohol composition of the present invention does not comprise alcohols having different carbon numbers. As used herein, the term “alcohols having different carbon numbers” means alcohols with different carbon chain lengths. For example, a mixture comprising a C₁₄ and a C₁₅ alcohol would be a mixture of alcohols having different carbon numbers.

Hydroformylation of the Cₙ₋₁ LAO Feed to Form a Cₙ Alcohol Mixture

While oxo hydroformylation imparts some branching, using a linear alpha olefin (LAO) feed does not yield an alcohol mixture with a branched alcohol content greater than the current achievable 30-40 wt %. The novel process of the present invention produces lightly branched alcohols showing selective branching, wherein the branching is more than 50%, even more than 80% and in further embodiments more than 90% at the 2-carbon position (the beta carbon) of the alcohol. As used herein, the term “selective branching” means that there is substantial branching at the 2-position of the alcohol. The term “selective branching” also encompasses branching at other positions of the carbon backbone of the alcohol. However, the total branching at these other positions of the carbon backbone is not more than 20%, preferably not more than 10%.

A further aspect of the invention relates to a process for making the Cₙ alcohol composition comprising linear and branched alcohols, wherein at least 80% of the branched alcohol chains are mono-branched chains with a branch at the second carbon atom. In a further embodiment, the process for making the Cₙ alcohol composition comprising linear and branched alcohols, wherein at least 85% of the branched alcohol chains are mono-branched chains with a branch at the second carbon atom. In an even further embodiment, the process for making the Cₙ alcohol composition comprising linear and branched alcohols, wherein at least 90% branched alcohol chains are mono-branched chains with a branch at the second carbon atom.

As discussed previously, isomerization of the linear alpha olefin feed may occur by methods well known to the person skilled in the art or by streaming the olefin feed over a zeolite catalyst at elevated temperature. The isomerized olefin feed is then subsequently used as the olefin feed for the hydroformylation step of the present invention. The conversion from olefin to aldehyde and alcohol can be achieved using hydroformylation technologies including low or high pressure cobalt organometallic catalyst or low pressure organometallic rhodium catalyst with or without modified ligands.

In one embodiment, a process for preparing a primary alcohol composition comprising linear and branched Cₙ alcohols, comprises:
a) isomerization of a C₍ₙ₋₁₎ linear alpha olefin feed to produce an C₍ₙ₋₁₎ isomerized olefin feed, wherein n is an odd integer, taking one or more values ranging from 11 to 21,
b) contacting the isomerized olefin feed with syngas and a hydroformylation catalyst,
c) hydrogenating the reaction mixture of step (b) and
d) harvesting the primary alcohol composition comprising linear and branched Cₙ alcohol chains, wherein at least 50% of the branched alcohol chains are mono-branched with a branch at the second carbon atom relative to the hydroxyl carbon of the Cₙ alcohol.

Isomerization of a C₍ₙ₋₁₎ linear alpha olefin feed to produce an C₍ₙ₋₁₎ isomerized olefin feed in the process for preparing a primary alcohol composition (step (a)) may yield an isomerized olefin feed rich in linear internal olefin (LIO) or an isomerized olefin feed rich in branched olefin (BO).

In one embodiment, the isomerization reaction in the process for preparing a primary alcohol composition yields an isomerized olefin feed rich in linear internal olefin.

In one embodiment, the isomerization reaction in the process for preparing a primary alcohol composition yields an isomerized olefin feed rich in branched olefin.

In a further embodiment, the isomerization of step (a) of the process for preparing a primary alcohol composition occurs by streaming the C₍ₙ₋₁₎ linear alpha olefin feed over an isomerization catalyst. In a further embodiment, the isomerization catalyst is a molecular sieve. In an even further embodiment, the isomerization catalyst is a zeolite. In an even further embodiment, the isomerization catalyst is an aluminosilicate zeolite catalyst. In a further embodiment, aluminosilicate zeolite catalyst is ZSM-48.

In one embodiment, the isomerization catalyst can be chosen from a family of zeolites, typically containing 10-membered rings, including but not limited to ZSM-48. Molecular sieves or zeolites are hydrated aluminosilicate minerals made from interlinked tetrahedra of alumina (AlO₄) and silica (SiO₄). In another embodiment, the isomerization catalyst comprises a molecular sieve. In a further embodiment, the isomerization catalyst comprises a zeolite. In a further embodiment, the isomerization catalyst is ZSM-48.

In embodiments if the invention, the SiO₂/Al₂O₃ ratio of the isomerization catalyst ranges from about 100 to about 75.

In a further embodiment, the SiO₂/Al₂O₃ ratio of the isomerization catalyst ranges from about 65 to about 75. In another embodiment, the SiO₂/Al₂O₃ ratio of the isomerization catalyst ranges from about 85 to about 95.

In one embodiment, the SiO₂/Al₂O₃ ratio of the ZSM-48 isomerization catalyst ranges from about 65 to about 75. In another embodiment, the SiO₂/Al₂O₃ ratio of the ZSM-48 isomerization catalyst ranges from about 85 to about 95.

In embodiments of the invention, the isomerization in step (a) is performed at a pressure ranging from about 1 to about 2 barg and a temperature ranging from about 130° C. to about 180° C. or from about 130° C. to about 160°, in a particular embodiment at 150° C.

In embodiments of the invention, the olefin feed in the step (a) isomerization is supplied at a weight hourly space velocity from about 1 to about 10 h⁻¹, or from 5 to 10 h⁻¹. In a particular embodiment, the weight hourly space velocity is about 5 h⁻¹. The inventors have found that a decrease in weight hourly space velocity results in increased skeletal isomerization with a higher yield in branched olefin and dimer and a lower yield of linear internal olefin.

In a further embodiment, the rate of conversion of the C₍ₙ₋₁₎ linear alpha olefin to the C₍ₙ₋₁₎ isomerized olefin in the step (a) isomerization is about 70 to about 90 percent.

In embodiments of the invention, the process for preparing a primary alcohol composition results in a Cₙ alcohol comprising linear and branched alcohol chains, wherein at least 80%, more preferably at least 90%, of the branched alcohol chains are mono-branched having a branch at the second carbon atom relative to the hydroxyl carbon.

In embodiments of the invention, the process for preparing a primary alcohol composition results in a Cₙ alcohol comprising linear and branched Cₙ alcohol chains, wherein the Cₙ alcohol composition is lightly branched, preferably having an average number of branches per molecule chain less than 1.4.

In embodiments of the invention, the process for preparing a primary alcohol composition comprises linear and branched Cₙ alcohols, wherein the average number of branches per molecule chain is greater than 0.4, alternatively greater than 0.6, yet optionally greater than 0.7.

In embodiments of the invention, the process for preparing a primary alcohol composition results in a C₁ alcohol comprising linear and branched C₁ alcohol chains, wherein the C₁ alcohol composition comprises less than 60 wt % linear alcohol, preferably less than 45 wt % linear alcohol, more preferably less than 5 wt % linear alcohol.

In embodiments of the invention, the hydroformylation organometallic catalyst in step (b) of the process for preparing a mixture of C₁ alcohols is a Group 9 transition metal. In a further embodiment, the Group 9 transition metal catalyst is cobalt or rhodium. In a further embodiment, the catalyst is an organometallic cobalt compound.

In a further embodiments of the invention, the ligand of the organometallic catalyst is a carbonyl or phosphine. In a further embodiment, the ligand of the organometallic catalyst is a carbonyl.

In embodiments of the invention, the hydroformylation organometallic catalyst is HCo(CO)₄.

Hydroformylation may be carried out by contacting the olefin feed with syngas (ranging from about 1:1 to 1.3:1 mixture of hydrogen:carbon monoxide) and a hydroformylation catalyst under the following reaction conditions: about 100 to about 140° C., pressure ranging from about 200 to about 400 bar and a hydroformylation catalyst concentration ranging from about 300 to about 3000 ppm.

Synthesis gas (syngas) is a mixture of carbon monoxide and hydrogen mainly produced from the steam reforming of methane and other light hydrocarbons from natural gas. It also is produced by gasification of coal and biomass.

In one embodiment, the syngas of step (b) of the process for preparing a primary alcohol composition comprises a mixture of hydrogen and carbon monoxide ranging from about 1:1 to about 1.3:1 hydrogen:carbon monoxide. In a further embodiment, the syngas of step (b) comprises a mixture of hydrogen and carbon monoxide with about 1.3:1 hydrogen to carbon monoxide ratio.

In one embodiment, the syngas of step (b) comprises a mixture of hydrogen and carbon monoxide ranging from about 1:1 to about 1.2:1 hydrogen:carbon monoxide. In a further embodiment, the syngas of step (b) comprises a mixture of hydrogen and carbon monoxide with about 1.2:1 hydrogen to carbon monoxide ratio.

In one embodiment, the syngas of step (b) comprises a mixture of hydrogen and carbon monoxide ranging from about 1:1 to about 1.1:1 hydrogen:carbon monoxide. In a further embodiment, the syngas of step (b) comprises a mixture of hydrogen and carbon monoxide with about 1.1:1 hydrogen to carbon monoxide ratio. In a further embodiment, the syngas of step (b) comprises a mixture of hydrogen and carbon monoxide with about 1.1 hydrogen to carbon monoxide ratio.

In one embodiment, the hydroformylation catalyst concentration ranges from about 500 to about 4000 ppm. In a further embodiment, the hydroformylation catalyst concentration ranges from about 500 to about 2500 ppm.

In embodiments of the invention, the hydroformylation temperature of step (b) ranges from about 80° C. to about 180° C. In a further embodiment, the hydroformylation temperature of step (b) ranges from about 100° C. to about 140° C. In a further embodiment, the processing temperature of step (b) is about 120° C.

In embodiments of the invention, the hydroformylation pressure of step (b) ranges from about 20 bar to about 320 bar. In a further embodiment, the hydroformylation pressure of step (b) ranges from about 100 bar to 320 bar. In a further embodiment, the processing temperature of step (b) is about 300 bar.

The resulting aldehyde reaction product from the hydroformylation step is then converted into an alcohol through a reduction process. Hydrogenation of the hydroformylation product yields the alcohol of the present invention. The reaction with hydrogen may occur in the presence of a hydrogenation catalyst. Suitable hydrogenation catalysts are transition metals such as Cr, Mo, W, Fe, Rh, Co, Ni, Pd, Pt, Ru, etc., or mixtures thereof, which may be applied to supports such as activated carbon or aluminum oxide, to increase the activity and stability.

The alcohol composition of the present invention can be isolated in pure form from the reaction mixture obtained from the hydrogenation by purification methods known to those skilled in the art, such as fractional distillation.

In another embodiment, the primary alcohol composition comprising linear and branched Cₙ alcohols, made from the following process:
a) isomerization of a C₍ₙ₋₁₎ linear alpha olefin feed to produce an C₍ₙ₋₁₎ isomerized olefin feed, wherein n is an odd integer, taking one or more values ranging from 11 to 21,
b) contacting the isomerized olefin feed with syngas and a hydroformylation catalyst,
c) hydrogenating the reaction mixture of step (b) and
d) harvesting the primary alcohol composition comprising linear and branched of Cₙ alcohol chains, wherein at least 50% of the branched alcohol chains are mono-branched chains with a branch at the second carbon atom relative to the hydroxyl carbon of the Cₙ alcohol.

In a further embodiment, the isomerization step (a) in the process for preparing a primary alcohol composition yields an isomerized olefin feed rich in linear internal olefin. In another embodiment, the isomerization step (a) in the process for preparing a primary alcohol composition yields an isomerized olefin feed rich in branched olefin.

Tridecanol

In one embodiment, a composition comprises linear and branched C₁₃ alcohols, wherein at least 50% of the branched alcohol chains are mono-branched chains with a branch at the second carbon atom relative to the hydroxyl carbon.

In a further embodiment, the tridecanol composition is characterized by at least 80% of the branched alcohol chains are mono-branched with a branch at the second carbon atom. In an even further embodiment, the tridecanol composition is characterized by at least 85% of the branched alcohol chains are mono-branched with a branch at the second carbon atom. In an even further embodiment, the tridecanol composition is characterized by at least 90% of the branched alcohol chains are mono-branched with a branch at the second carbon atom.

In another embodiment, the average degree of branching in the tridecanol composition is less than 1.4. In a further embodiment, the average degree of branching in the tridecanol composition is less than 1 and greater than 0.4.

Pentadecanol

In different embodiments of the invention, a composition comprises linear and branched C₁₅ alcohol chains, wherein at least 50% of the branched alcohol chains are mono-branched chains with a branch at the second carbon atom relative to the hydroxyl carbon.

In a further embodiment, the pentadecanol composition is characterized by at least 80% branched alcohol chains are mono-branched with a branch at the second carbon atom. In an even further embodiment, the pentadecanol composition is characterized by at least 85% branched alcohol chains are mono-branched with a branch at the second carbon atom. In an even further embodiment, the pentadecanol composition is characterized by at least 90% branched alcohol chains are mono-branched with a branch at the second carbon atom.

In embodiments of the invention, the average degree of branching in the pentadecanol composition is less than 1.4. In a further embodiment, the average degree of branching in the pentadecanol composition is less than 1 and greater than 0.4. In another embodiment, the average degree of branching in the pentadecanol composition is about 0.5 to about 0.8.

In embodiments of the invention, the viscosity of the pentadecanol composition is about 8 to about 9 cSt. In embodiments of the invention, the density of the pentadecanol composition is about 6.5 to about 7.2 g/cm³. In embodiments of the invention, the melting point of the pentadecanol composition is about 20 to about 30° C.

Physical and chemical properties of three pentadecanol products produced in batch-mode operation from C₁₄ olefin feeds (LAO feed and isomerized feed) were tested. Gas chromatography and NMR analysis confirmed the structural differences between the pentadecanol samples obtained from LAO-feed and those obtained from isomerized feed. The pentadecanol samples obtained from isomerized feed showed an increased average degree of branching and displayed a significantly higher branched alcohol content as compared to the alcohol generated from the LAO feed. Surprisingly, the pentadecanols made from isomerized feed showed almost exclusively branching at the second carbon (beta carbon) position of the chain. Furthermore, based on the branch-site distribution, the inventors found that the pentadecanols made of isomerized feed had a total of less than 10% branches at 3-, 4-, both 3- and 4- and 5+ carbon positions of the chain.

Therefore, it was concluded that pentadecanols made from isomerized feed according to the method of the invention show a highly selective branch-site distribution. Without wishing to be bound by theory, it is believed that alcohol chains comprising predominantly mono-branched chains with the branch in the second carbon position have a direct influence in on the molecular packing shape of the surfactants obtained therefrom, in particular the branch at the second carbon position tends to produce cylindrical or inverse cone shape packing, characteristic for the more hydrophobic surfactants. The method of the invention allows tailoring the geometry of the alcohol molecular chain which has an impact on the behavior of the derivatives obtained therefrom.

The various descriptive elements and numerical ranges disclosed herein for the reactants used to make the C₁₁-C₂₁ alcohols, and their use can be combined with other descriptive elements and numerical ranges to describe the invention(s); further, for a given element, any upper numerical limit can be combined with any lower numerical limit described herein. The features of the invention are described in the following non-limiting examples.

### Examples

Gas Chromatography Procedures

Liquid samples from the reactor effluent were analyzed on an Agilent 7890 Gas Chromatograph (GC) equipped with FID detectors and automatic liquid samplers (ALS). Three GC methods were employed to analyze the samples—one for measuring the linear alpha olefin (LAO) content, the second for measuring the branched olefin (BO) content of the feeds and the third for measuring the composition of the alcohol. The typical injection size for all methods was about 0.2 μl.

The first GC method for determining the LAO content was as follows. The GC column used was an Agilent DB-WAX (60 m×250 μm×0.2.5 μm) column. The GC was operated in constant flow mode at 40 psi (280 kPa) inlet pressure and with column flow of 1.839 mL/min using helium as a carrier gas. The following oven procedure was used: Initial temperature of 140° C., hold for 17 minutes; ramp at 25° C./min to 240° C. and hold for 8 minutes. Total analysis time was approximately 29 minutes.

The second GC method for determining the branched olefin (BO) content was as follows. The liquid sample was first fully hydrogenated to saturated material, from which the BO content was determined by analyzing the total branched material. The column used was Agilent HP-1 (60 m×2.50 μm×1 μm) and the inlet liner was a split inlet liner (obtained from Agilent) that was pre-packed with 1 cm height 1% Pt/Al₂O₃. The GC was operated in ramped pressure mode with an initial pressure of 20 psi (140 kPa) to 50 psi (340 kPa) at 7 psi/min (50 kPa/min) using hydrogen as a carrier gas. The following oven procedure was used: Initial temperature of 140° C., hold for 17 minutes, ramp at 25° C./min to 240° C. and hold for 8 minutes. Total analysis time was about 29 minutes.

The linear internal olefin (LIO) content (wt %) of the isomerized feed may be determined as follows: LIO=100−LAO content (wt %) of the isomerized feed−BO content (wt %) of the isomerized feed.

The third GC method for measuring the composition of the alcohol was as follows. To determine the weight fraction of branched alcohols, the region of the GC spectrum for a given carbon number alcohol was split between the normal alcohol such as n-pentadecanol and the respective branched alcohols. The region eluting immediately before a particular normal alcohol is assigned to the corresponding branched alcohols with no differentiation to the type of branching.

Nuclear-Magnetic Resonance (NMR) Procedures

Two NMR methods were employed to analyze the samples: ¹H solution-state NMR was used to determine the degree of branching or the branching index (BI); ¹³C solution-state NMR was used to determine the branch-site distribution, i.e. the percentage of branching in the 2-, 3-, 4- and 5⁺-positions of the alcohol molecule (herein below denoted with b2, b3, b4, b5+).

Quantitative ¹H and ¹³C NMR spectra were recorded in the solution-state using a Bruker Avance III 600 NMR spectrometer operating at 600.21 and 150.92 MHz respectively. All spectra were recorded using a ¹³C optimized dual ¹³C-¹H direct-detection extended-temperature 10 mm cryo-probehead at 300 K (26.85° C.) using nitrogen gas for all pneumatics.

Approximately 500 pf of material was dissolved in approximately 2 ml of solvent with relaxation agent consisting of 50 mM chromium-(III)-acetylacetonate in chloroform-d.

For ¹H NMR acquisition standard single pulse excitation was employed using a 300 tip angle based on a 17 μs 90° pulse, a 15 s relaxation delay and 10 Hz sample rotation. A total of 94 k data points were acquired per FID with a dwell time of 39.6 μs resulting in an acquisition time of 3.8 s and a spectral window of 13 kHz (21 ppm). A total of 12 transients were recorded per spectrum using a digital filter optimized for the cryo-probehead (DIGMOD=baseopts). The FID was zero-filled to 128 k data points, an exponential window function applied with 0.3 Hz line-broadening. This setup was chosen primarily for quantitative results for a wide range of materials. Quantitative ¹H NMR spectra were processed, integrated and quantitative properties determined using custom spreadsheets. All chemical shifts was indirectly referenced to TMS at 0 ppm using the residual protonated solvent signal at 7.26 ppm.

For ¹³C NMR acquisition standard single pulse excitation was employed using a 900 tip angle using a 10.5 μs pulse, no NOE, a 10 s relaxation delay, bi-level WALTZ16 decoupling scheme and 10 Hz sample rotation. A total of 45 k data points were acquired per FID with a dwell time of 16.2 μs resulting in an acquisition time of 4.2 s and a spectral window of 31 kHz (205 ppm). A total of 512 transients were recorded per spectrum using 4 dummy scans and a digital filter optimized for the cryo-probehead (DIGMOD=baseopts). The FID was zero-filled to 128 k data points, an exponential window function applied with 5 Hz line-broadening. This setup was chosen primarily for quantitative results for a wide range of materials. Quantitative ¹³C{¹H} NMR spectra were processed, integrated and quantitative properties determined using custom spreadsheets. All chemical shifts was indirectly referenced to TMS at 0 ppm using the central signal of the deuterated solvent at 77.0 ppm.

The ¹H NMR spectra showed characteristic signals corresponding to the presence of higher alcohols with branched aliphatic chains. Methylene protons adjacent to the hydroxyl group (CH2a) were observed and integrated between 3.9-3.0 ppm. The remaining aliphatic and hydroxyl protons (CHn+OH) were observed and integrated between 2.0-0.5 ppm. The methyl protons (CH3) were observed and integrated between 1.0-0.5 ppm.

The average number of branches per molecule of the mixture, as determined by ¹H NMR, was calculated using:

BrM=((2*CH3)/(3*CH2a))−1

The ¹³C{¹H} NMR spectra showed characteristic signals corresponding to the presence of higher alcohols with branched aliphatic chains as well as linear alcohol. The relative location of the branch site along the chain with respect to the hydroxyl group could be distinguished based upon the characteristic chemical shifts of the methylene sites directly adjacent to the hydroxyl group (CH2a) at carbon 1.

Alcohols with branches at carbon 2 (CH2aB2) were observed and integrated between 72.3-63.6 ppm. Similarly alcohols with branches at carbon 3 and 5 or more were observed and integrated between 60.8-58.0 and 62.6-62.0 ppm respectively. Alcohols with branches at both carbons 3 and 4 (CH2aB34) were observed and integrated between 62.0-60.8 ppm. Linear alcohols (CH2aB0) were observed and integrated between 63.6-62.6 ppm. The bulk aliphatic signal (CHn) was observed and integrated between 55.0-5.0 ppm.

The total amount of all methylene sites direction adjacent to the hydroxyl group (CH2aB) was determined as the sum of each resolved alcohol type:

CH2aB=CH2aB2+CH2aB3+CH2aB5+CH2aB34+CH2aB0

The relative content of linear and distinguished type of branched alcohol was calculated with respect to the total amount of alcohol and given in mole percent:

mol % Bn=100*CH2aBn/CH2aB

The following examples illustrate the effect of the olefin feed to the type and amount of branching of the alcohol product.

### Example 1

Isomerization of a C₁₄ linear alpha olefin feed was performed by methods well known to the person skilled in the art or by streaming the olefin feed over a zeolite catalyst at elevated temperature. The zeolite catalyst can be chosen from a family of zeolites, typically containing 10-membered rings, including but not limited to ZSM-48.

Isomerization was conducted under the following reaction conditions (Table 1): temperature from 130° C. to 160° C., pressure 1.5 barg and 5-10 h⁻¹ weight hourly space velocity (WHSV).

TABLE 1
Process conditions, conversion range and isomerized olefin content
for the isomerization reaction.

| | | | | | Internal | Branched |
|---|---|---|---|---|---|---|
| | | | | | olefin | Olefin |
| | | Pressure | WHSV | Conversion | content | content |
| Feed | T(° C.) | (barg) | (h⁻¹) | range (%) | (wt %) | (wt %) |
| Isomerized | 140- | 1.5 | 10 | 70-90 | 70-90 | 0-20 |
| Feed I | 150 | | | | | |
| Isomerized | 140- | 1.5 | 5 | 70-90 | 40-80 | 10-50 |
| Feed II | 150 | | | | | |

Two gas chromatographic (GC) methods were employed to characterize the olefin feeds: one for measuring the linear alpha olefin content (LAO) and the second for measuring the branched olefin (BO) content of the feed. The GC data are disclosed in FIG. 1. Specifically, FIG. 1 discloses the on stream composition of two isomerized C₁₄ olefin feeds showing the conversion of linear alpha olefin to linear internal olefin, branched olefin and dimer. The circles of FIG. 1 are directed to the conversion of LAO, the triangles are directed to the yield of the linear internal olefin (LIO), the squares are directed to the yield of the branched olefin and the diamonds are directed to the yield of the dimer.

The reaction conditions used to produce the isomerized Feed I olefin feed from the LAO feed were: about 140° C., about 1.5 barg and about 10 h⁻¹ weight hourly space velocity. The reaction conditions used to produce the isomerized Feed II olefin feed from the LAO feed were: about 150° C., about 1.5 barg and about 5 h⁻¹ weight hourly space velocity. A further decrease in weight hourly space velocity results in increased skeletal isomerization with a higher yield in branched olefin and dimer and a lower yield of linear internal olefin.

Under these isomerization conditions, Feed I displayed a linear internal olefin content of 70 to 90 wt % and a branched olefin content of 0 to 20 wt %. Feed II displayed a linear internal olefin content of 40 to 80 wt % and a branched olefin content of 10 to 50 wt %. Conversion from the LAO feed to the mixture of internal and branched olefins was 70-90% for both Feed I and Feed II.

FIG. 1 shows that the on stream composition of the olefin changed with time on stream (TOS). Specifically, the LIO content of the C₁₄ olefin feed increased and the branched olefin content decreased after 600 hours on stream when the WHSV was increased.

### Example 2

The isomerized olefin feed of Example 1 (Feed I and Feed II) was used as the olefin feed for the hydroformylation reaction. The conversion from olefin to aldehyde and alcohol can be achieved using well known hydroformylation technologies including low or high pressure cobalt organometallic catalyst or low pressure organometallic rhodium catalyst with or without modified ligands.

Hydroformylation was carried out by contacting the olefin feed with syngas, a mixture comprising a range from about 1:1 to about 1.3:1 hydrogen:carbon monoxide, and a HCo(CO)₄ hydroformylation catalyst under the following reaction conditions: temperature ranging from about 100 to about 140° C., pressure ranging from 200 to 320 bar and a hydroformylation catalyst concentration ranging from about 300 to 2500 ppm.

Three C₁₄ olefin feeds were used in the hydroformylation reactions. The first olefin feed was a C₁₄ LAO feed, the second feed was isomerized olefin Feed I and the third feed was isomerized olefin Feed II. The organometallic catalyst was HCo(CO)₄. The hydroformylation reaction conditions for the three C₁₄ olefin feeds are shown in Table 2:

TABLE 2
Hydroformylation reaction: feed and process conditions

| | | | Cobalt concentration. |
|---|---|---|---|
| Feed | T(° C.) | Pressure (bar) | (ppm) |
| C₁₄ LAO | 120 | 300 | 500 |
| C₁₄ isomerized Feed I | 120 | 300 | 500 |
| C₁₄ isomerized Feed II | 120 | 300 | 2000 |

### Example 3

Characterization of the physical and chemical properties of the three pentadecanol products produced in batch-mode operation from the olefin feeds of Example 1 are summarized in Table 3.

Three pentadecanol mixtures were produced in batch-mode operation through high-pressure Cobalt hydroformylation and subsequent hydrogenation of three different tetradecene olefin feeds: C₁₄ LAO, and two isomerized, branched C₁₄ olefins (Feed I and Feed II; see FIG. 1). Feeds I and II were derived from isomerizing C₁₄ LAO over a ZSM-48-based catalyst at two different reaction conditions.

Gas chromatography and NMR (¹H and ¹³C) analysis confirmed the structural differences between the alcohol samples, reflected in the increased average degree of branching of the alcohol compositions based on the isomerized olefin feed. For all samples the branching was almost exclusively in the 2-position. Gas chromatography was employed to determine the composition of the alcohol. ¹H NMR was employed to determine the degree of branching and ¹³C NMR was employed to determine the percent branching in the 2-, 3-, 4- and 5⁺-carbon positions of the alcohol.

Linear and branched content of the three alcohol compositions as well as average degree of branching and physical properties are shown in Table 3. Gas chromatography analysis of the pentadecanol mixtures showed that the feed composition with respect to the percentage of linear alpha olefins, linear internal olefins and branched olefins significantly altered the alcohol properties.

The hydroformylation of the isomerized olefin feed resulted in a significantly reduced proportion of linear alcohols in the final product and an increased average number of branches per molecule for the resulted pentadecanol: 0.6 (pentadecanol from Feed I) and 0.8 (pentadecanol from Feed II) as compared to 0.4 for the pentadecanol from LAO feed. The linear alcohol content and branched alcohol content of the pentadecanol products obtained from the LAO-feed and from the isomerized feeds (Feed I and Feed II) are shown in Table 3. The pentadecanols obtained from isomerized feed contain lower percentage of linear alcohol than the pentadecanols obtained from LAO-feed. All products are pure alcohols (pentadecanols) obtained from the C14 olefins (isomerized or not) therefore the values in mol % and wt % are equivalent.

TABLE 3
Physical and chemical properties of pentadecanol products produced in
batch-mode operation from C14 olefin feeds:

| | Linear | Branched | BI | υ @ | p @ | |
|---|---|---|---|---|---|---|
| | alcohol | alcohol | (branches/ | 25° C. | 60° C. | Tₘₚₜ |
| Feed | (mol %) | (mol %) | molecule) | (cSt) | (gcm³) | (° C.) |
| C₁₄ LAO | 65.7 | 34.3 | 0.4 | 8.62 | 6.99 | 35 |
| Isomerized | 57.0 | 43.0 | 0.6 | 8.37 | 6.78 | 26 |
| C₁₄ (Feed I) | | | | | | |
| Isomerized | 42.6 | 57.4 | 0.8 | 8.32 | 6.75 | 24 |
| C₁₄ (Feed II) | | | | | | |

Table 4 summarizes the branch-site distribution as determined using ¹³C-NMR for the pentadecanol samples. The pentadecanol made from the isomerized feed according to the method of the invention (Feed I and Feed II) displayed a higher branched alcohol content as compared to the alcohol generated from the LAO feed: 43.0 mol branched pentadecanol (Feed I) and 57.4 mol % branched pentadecanol (Feed II) as compared to 34.3 mol % branched pentadecanol (LAO-feed). Surprisingly, the branching was predominantly at the second carbon position (b2) of the pentadecanol: 92.7 0% and 99.0 00 of the branched alcohol chains were branched at the second carbon position (b2) for the pentadecanols produced from isomerized Feed II and, respectively, from isomerized Feed I.

TABLE 4
Branch-site distribution of pentadecanol products produced in batch-
mode operation from C₁₄ olefin feeds:

| | Branched | | | | | | |
|---|---|---|---|---|---|---|---|
| | alcohol | -b2 | -b3* | | | b2/ | b3, 4, |
| | (Br) | (mol | (mol | -b3, 4 | -b5⁺* | Br | 5⁺/Br |
| Feed | (mol %) | %) | %) | (mol %) | (mol %) | (%) | (%) |
| C₁₄ LAO | 34.3 | 33.1 | — | 1.2 | — | 96.5 | 3.4 |
| Isomerized | 43.0 | 42.6 | — | 0.4 | — | 99.0 | 0.9 |
| C₁₄ (Feed I) | | | | | | | |
| Isomerized | 57.4 | 53.2 | — | 4.1 | — | 92.7 | 7.1 |
| C₁₄ | | | | | | | |
| (Feed II) | | | | | | | |

*not observed

As shown in Table 4, no measurable evidence was found for branched chains having a branch at third and fifth carbon position or higher (b3, b5⁺). The percentage of branched chains having a branches at both the third and the fourth carbon position (b3,4) is low for all pentadecanols: 0.9% for pentadecanols from Feed I and, respectively, 7.1% for pentadecanols from Feed II as compared with 3.4% for pentadecanols from LAO-feed. The high percentage of branched chains having a branch in the second position demonstrates that the method of the invention selectively forms this structure, in a controlled and reproducible manner.

### Additional Embodiments

This disclosure may further include one or more of the following non-limiting embodiments:

E1. A primary alcohol composition comprising linear and branched Cn alcohol chains, wherein at least 50% of the branched alcohol chains are mono-branched chains with a branch at the second carbon atom relative to the hydroxyl carbon, where n is an odd integer, taking one or more values ranging from 11 to 21.

E2. The composition of E1, wherein at least 80% of the branched alcohol chains are mono-branched chains with a branch at the second carbon atom.

E3. The composition of E1, wherein at least 90% of the branched alcohol chains are mono-branched chains with a branch at the second carbon atom.

E4. The composition of any of E1 to E3, wherein the composition is lightly branched having an average number of branches per molecule chain that is less than 1.4.

E5. The composition of any of E1 to E4, wherein the average number of branches per molecule chain is greater than 0.4, optionally greater than 0.6, yet optionally greater than 0.7.

E6. The composition of any of E1 to E5, comprising less than 60 wt % linear alcohol, preferably less than 45 wt % linear alcohol, more preferably less than 5 wt % linear alcohol.

E7. The composition of any of E1 to E6, wherein n is equal to 11, 13, 15, 17, 19 or 21.

E8. The composition of any of E1 to E7, wherein the linear and branched Cn alcohol is converted from isomerized a C(n−1) linear alpha olefin.

E9. A process for preparing a primary alcohol composition comprising linear and branched Cn alcohols, comprising:
a) isomerization of a C(n−1) linear alpha olefin feed to produce an C(n−1) isomerized olefin feed, wherein n is an odd integer, taking one or more values ranging from 11 to 21,
b) contacting the isomerized olefin feed with syngas and a hydroformylation catalyst,
c) hydrogenating the reaction mixture of step (b) and
d) harvesting the primary alcohol composition comprising linear and branched Cn alcohol chains, wherein at least 50% of the branched alcohol chains are mono-branched chains with a branch at the second carbon atom relative to the hydroxyl carbon of the Cn alcohol.

E10. The process of E9, wherein the isomerization reaction yields an isomerized olefin feed rich in linear internal olefin.

E11. The process of E9, wherein the isomerization reaction yields an isomerized olefin feed rich in branched olefin.

E12. The process of any of E9 to E11, wherein the isomerization of step (a) is performed by streaming the C(n−1) linear alpha olefin feed over an isomerization catalyst.

E13. The process of E12, wherein the isomerization catalyst comprises a molecular sieve, preferably comprises a zeolite, more preferably the isomerization catalyst is ZSM-48.

E14. The process of E13, wherein the SiO2/Al2O3 ratio of the zeolite isomerization catalyst ranges from about 100 to about 75.

E15. The process of any of E9 to E14, wherein isomerization in step (a) is performed at a pressure ranging from about 1 to about 2 barg and a temperature ranging from about 130° C. to about 160° C.

E16. The process of any of E9 to E15, wherein the linear alpha olefin feed in step (a) is supplied at a weight hourly space velocity from about 5 to about 10 h-1.

E17. The process of any of E9 to E16, wherein the rate of conversion of the C(n−1) linear alpha olefin to the C(n−1) isomerized olefin in step (a) is about 70 to about 90 percent.

E18. The process of any of E9 to E17, wherein at least 80%, more preferably at least 90%, of the branched alcohol chains are mono-branched having a branch at the second carbon atom relative to the hydroxyl carbon of the Cn alcohol.

E19. The process of any of E9 to E18, wherein the Cn alcohol composition is lightly branched having an average number of branches per molecule chain less than 1.4.

E20. The process of any of E9 to E19, wherein the average number of branches per molecule chain is greater than 0.4, optionally greater than 0.6, yet optionally greater than 0.7.

E21. The process of any of E9 to E20, wherein the Cn alcohol composition comprises less than 60 wt % linear alcohol, preferably less than 45 wt % linear alcohol, more preferably less than 5 wt % linear alcohol.

E22. A composition comprising one or more derivatives of the primary alcohol composition of any of E1 to E8 or a primary alcohol composition obtainable by the process of any of E9 to E21.

E23. The composition of E22, wherein the derivative comprises esters of dicarboxylic acids, esters of polycarboxylic acids, alkoxylated alcohols, sulfated alcohols, sulfated alkoxylated alcohols and alcohol ether amines.

E24. The composition of E22 wherein the derivative comprises esters of the primary alcohol composition with one or more acids.

E25. The composition of E24, wherein the acids comprise one or more of phthalic acid, adipic acid, sebacic acid, lauric acid, myristic acid, palmitic acid, stearic acid, oleic acid, succinic acid and trimellitic acid.

E26. The composition of E22, wherein the derivative comprises phosphites of low volatility to be used as polymer stabilizers.

In the specification and in the claims, the terms “including” and “comprising” are open-ended terms and should be interpreted to mean “including, but not limited to”. These terms encompass the more restrictive terms “consisting essentially of” and “consisting of” It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

All documents described herein are incorporated by reference herein, including any priority documents and/or testing procedures to the extent they are not inconsistent with this text. As is apparent from the foregoing general description and the specific embodiments, while forms of the present disclosure have been illustrated and described, various modifications can be made without departing from the spirit and scope of the present disclosure. Accordingly, it is not intended that the present disclosure be limited thereby.

1. A primary alcohol composition comprising linear and branched Cₙ alcohol chains, wherein at least 50% of the branched alcohol chains are mono-branched chains with a branch at the second carbon atom relative to the hydroxyl carbon, where n is an odd integer, taking one or more values ranging from 11 to 21.

2. The composition of claim 1, wherein at least 80% of the branched alcohol chains are mono-branched chains with a branch at the second carbon atom.

3. The composition of claim 1, wherein at least 90% of the branched alcohol chains are mono-branched chains with a branch at the second carbon atom.

4. The composition of claim 1, wherein the composition is lightly branched having an average number of branches per molecule chain that is less than 1.4.

5. The composition of claim 1, wherein the average number of branches per molecule chain is greater than 0.4, optionally greater than 0.6, yet optionally greater than 0.7.

6. The composition of claim 1, comprising less than 60 wt % linear alcohol, preferably less than 45 wt % linear alcohol, more preferably less than 5 wt % linear alcohol.

7. The composition of claim 1, wherein n is equal to 11, 13, 15, 17, 19 or 21.

8. The composition of claim 1, wherein the linear and branched Cₙ alcohol is converted from isomerized a C₍ₙ₋₁₎ linear alpha olefin.

9. A process for preparing a primary alcohol composition comprising linear and branched Cₙ alcohols, comprising:
a) isomerization of a C₍ₙ₋₁₎ linear alpha olefin feed to produce an C₍ₙ₋₁₎ isomerized olefin feed, wherein n is an odd integer, taking one or more values ranging from 11 to 21,
b) contacting the isomerized olefin feed with syngas and a hydroformylation catalyst,
c) hydrogenating the reaction mixture of step (b) and
d) harvesting the primary alcohol composition comprising linear and branched Cₙ alcohol chains, wherein at least 50% of the branched alcohol chains are mono-branched chains with a branch at the second carbon atom relative to the hydroxyl carbon of the Cₙ alcohol.

10. The process of claim 9, wherein the isomerization reaction yields an isomerized olefin feed rich in linear internal olefin.

11. The process of claim 9, wherein the isomerization reaction yields an isomerized olefin feed rich in branched olefin.

12. The process of claim 9, wherein the isomerization of step (a) is performed by streaming the C₍ₙ₋₁₎ linear alpha olefin feed over an isomerization catalyst.

13. The process of claim 12, wherein the isomerization catalyst comprises a molecular sieve, preferably comprises a zeolite, more preferably the isomerization catalyst is ZSM-48.

14. The process of claim 13, wherein the SiO₂/Al2O₃ ratio of the zeolite isomerization catalyst ranges from about 100 to about 75.

15. The process of claim 9, wherein isomerization in step (a) is performed at a pressure ranging from about 1 to about 2 barg and a temperature ranging from about 130° C. to about 160° C.

16. The process of claim 9, wherein the linear alpha olefin feed in step (a) is supplied at a weight hourly space velocity from about 5 to about 10 h⁻¹.

17. The process of claim 9, wherein the rate of conversion of the C₍ₙ₋₁₎ linear alpha olefin to the C₍ₙ₋₁₎ isomerized olefin in step (a) is about 70 to about 90 percent.

18. The process of claim 9, wherein at least 80%, more preferably at least 90%, of the branched alcohol chains are mono-branched having a branch at the second carbon atom relative to the hydroxyl carbon of the Cₙ alcohol.

19. The process of claim 9, wherein the Cₙ alcohol composition is lightly branched having an average number of branches per molecule chain less than 1.4.

20. The process of claim 9, wherein the average number of branches per molecule chain is greater than 0.4, optionally greater than 0.6, yet optionally greater than 0.7.

21. The process of claim 9, wherein the Cₙ alcohol composition comprises less than 60 wt % linear alcohol, preferably less than 45 wt % linear alcohol, more preferably less than 5 wt % linear alcohol.

22. A composition comprising one or more derivatives of the primary alcohol composition of claim 1.

23. The composition of claim 22, wherein the derivative comprises esters of dicarboxylic acids, esters of polycarboxylic acids, alkoxylated alcohols, sulfated alcohols, sulfated alkoxylated alcohols and alcohol ether amines.

24. The composition of claim 22 wherein the derivative comprises esters of the primary alcohol composition with one or more acids.

25. The composition of claim 24, wherein the acids comprise one or more of phthalic acid, adipic acid, sebacic acid, lauric acid, myristic acid, palmitic acid, stearic acid, oleic acid, succinic acid and trimellitic acid.

26. The composition of claim 22, wherein the derivative comprises phosphites of low volatility to be used as polymer stabilizers.
